(19) United States
(12) Patent Application Publication
(10) Pub. No.: US 2021/0090689 A1
(43) Pub. Date: Mar. 25, 2021

(54) METHODS FOR PREDICTING THE GIBBS FREE ENERGY OF BIOCHEMICAL REACTIONS

(71) Applicant: KING ABDULLAH UNIVERSITY OF SCIENCE AND TECHNOLOGY, Thuwal (SA)

(72) Inventors: Xin GAO, Thuwal (SA); Meshari Saud ALAZMI, Thuwal (SA); Hiroyuki KUWAHARA, Thuwal (SA)

(21) Appl. No.: 16/967,498

(22) PCT Filed: Jan. 8, 2019
(86) PCT No.: PCT/IB2019/050136

§ 371 Date: Aug. 5, 2020

Related U.S. Application Data
(60) Provisional application No. 62/641,610, filed on Mar. 12, 2018.

(51) Int. Cl. G16C 20/30; G16C 20/10; G16C 20/70; G06F 17/18; G16B 40/20

(52) U.S. Cl. CPC G16C 20/30; G16C 20/10; G16B 40/20; G06F 17/18; G16C 20/70

(57) ABSTRACT

Embodiments of the present disclosure describe a fingerprint contribution method for predicting a Gibbs free energy of biochemical reactions, methods of training a fingerprint contribution model for predicting a Gibbs free energy of biochemical reactions, methods of predicting a Gibbs free energy of biochemical reactions, a non-transitory computer readable medium comprising instructions which, when read by a computing device, cause a processor to execute a method for predicting a Gibbs free energy of biochemical reactions, and the like.

### CROSS-REFERENCE TO RELATED APPLICATIONS

This application claims priority to U.S. Provisional Patent Application No. 62/641,610, filed on Mar. 12, 2018, entitled “FC: A NOVEL METHOD TO ESTIMATE GIBBS FREE ENERGY OF BIOCHEMICAL REACTIONS,” the disclosure of which is incorporated herein by reference in its entirety.

### BACKGROUND

A number of computational methods have been proposed for the prediction of Gibbs free energy of biochemical reactions. The most commonly applied ones are variants of the group contribution method. The group contribution method is a linear regression method which uses predefined 2D substructures as features and estimates the Gibbs free energy of formation of a compound by the sum of the weight of substructure fragments into which this compound can be decomposed. Because these substructure fragments can be combined to compose molecules that are not seen in the training set, the group contribution method has a potential to cover a wide range of biochemical reactions. The main challenge of the group contribution method is to identify useful 2D substructure fragments that can be used as its features. The manual selection of such substructure fragments is a complex, tedious, and time-consuming task requiring expert knowledge on the structure-activity-relationship (SAR) of metabolites. This is because the selection of substructure features needs to satisfy two objectives: (i) to compose all compounds in the training set by the substructure fragments and (ii) to have useful substructure fragments for the biochemical thermodynamic prediction. Furthermore, since the selection of these features depends strongly on the compounds present in the training set, its decision also needs to take into account the characterization of the 2D structure of unseen compounds so as to avoid severely limited prediction coverage.

### SUMMARY

In general, embodiments of the present disclosure describe systems and methods for predicting a Gibbs free energy of biochemical reactions, and the like.

Embodiments of the present disclosure describe a method of predicting standard reaction Gibbs free energies of biochemical reactions comprising acquiring a plurality of biochemical reactions involving chemical compounds having concrete 2D structures; generating chemical fingerprints and/or molecular descriptors to represent the chemical compounds involved in the plurality of biochemical reactions; and feeding the plurality of biochemical reactions and the chemical fingerprints and/or molecular descriptors, as inputs, to a trained fingerprint contribution model to obtain an output in the form of predicted standard reaction Gibbs free energies of the plurality of biochemical reactions.

Embodiments of the present disclosure further describe a method of training a fingerprint-contribution model for predicting a standard Gibbs free energy of biochemical reactions comprising acquiring a training set comprising a plurality of biochemical reactions and Gibbs free energies of reaction that correspond to the plurality of biochemical reactions; forming a pool in which chemical fingerprint- and/or molecular descriptor-based features represent each chemical compound that participates in the plurality of biochemical reactions provided in the training set; applying a systematic feature selection procedure to reduce the pool to a subset of relevant chemical fingerprint- and/or molecular descriptor-based features; and applying a regularized linear regression method to the training set to construct the fingerprint contribution model.

Embodiments of the present disclosure describe a non-transitory computer readable medium comprising instructions which, when read by a computing device, cause a processor to execute a method for predicting a standard Gibbs free energy of biochemical reactions, the method comprising (a) loading a trained fingerprint contribution model into a program memory of the computing device; and (b) feeding a dataset comprising chemical fingerprint-based and/or molecular descriptor-based features into the trained fingerprint contribution model to obtain an output, wherein the chemical fingerprint- and/or molecular descriptor based features represent chemical compounds present in a plurality of biochemical reactions and the output comprises predicted standard reaction Gibbs free energies for the plurality of biochemical reactions.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a flowchart of a method of training a fingerprint-contribution model for predicting a Gibbs free energy (e.g., standard reaction Gibbs free energy) of biochemical reactions, according to one or more embodiments of the present disclosure.

FIG. 2 is a flowchart of a method of predicting Gibbs free energy of reaction (e.g., standard reaction Gibbs free energy) for biochemical reactions, according to one or more embodiments of the present disclosure.

FIG. 3 is a flowchart of a method of predicting a standard Gibbs free energy of biochemical reactions using a non-transitory computer medium comprising instructions which, when ready by a computing device, cause a processor to execute the method, according to one or more embodiments of the present disclosure.

FIG. 4 is a diagrammatic representation of a machine in the example form of a computer system 400 within which a set of instructions may be executed causing the machine to perform any one or more of the methods, processes, operations, applications, or methodologies discussed herein, according to one or more embodiments of the present disclosure.

FIGS. 5A-5C are heatmaps showing the classification of the weight of selected chemical fingerprint and molecular descriptor features based on the leave-on-out cross-validation analysis of the lasso model, according to one or more embodiments of the present disclosure.

FIGS. 6A-6C show hyperparameters based on the leave-one-out cross-validation (LOOCV) results of the ridge regression: (A) shows a grid search of hyperparameters, where each cell shows the mean absolute error (MAE) from the LOOCV results for a specified combination of the zero count threshold θ and the ridge regularization parameter λ_{ridge}, and the color of each cell indicates the ranking of its prediction performance based on the mean absolute error; (B) shows a refinement of the value of λ_{ridge} with a fine increment while fixing θ=14; (C) shows the number of selected features for each feature filtering step, with θ=14, the number of the selected features turn out to be 223 (the unit of the LOOCV MAE is kJ/mol), according to one or more embodiments of the present disclosure.

FIGS. 7A-7D is a comparison of the results from the leave-one-out cross-validation (LOOCV), according to one or more embodiments of the present disclosure. The left pane shows scatterplots in which the observed values for the standard Gibbs energy (x-axis) and the predicted values (y-axis) are compared. The left pane also shows, for each model, the Pearson correlation coefficient (r) between the observed data and the predicted data. The right pane displays the distribution of the absolute error computed for each pair of observed and predicted values. In the right pane, the x-axis uses a base-10 log scale and the mean prediction error (μ) is shown for each model. (A) shows the fingerprint-contribution (FC) model; (B) shows the component-contribution (CC) model; (C) shows the group-contribution (GC) model; and (D) shows the reaction-contribution (RC) model. The unit of ΔG₀ is kJ/mol.

FIG. 8 includes boxplots showing how the distribution of the absolute errors is partitioned for in-range samples and out-of-range reaction samples in each model, according to one or more embodiments of the present disclosure. In each boxplot, the number shown in the upper side of the plot indicates the sample size, while the diamond-shaped point represents the mean absolute error (MAE). Open circle points represent outliers which are defined to be those samples that are outside of the range between the lower quartile minus 1.5 times the interquartile distance (IQD) and the upper quartile plus 1.5 times the IQD. The unit of absolute error is kJ/mol.

FIGS. 9A-9B are graphical views illustrating estimation of the prediction accuracy for the KEGG reactions by each model trained by the Noor et al.-based dataset, according to one or more embodiments of the present disclosure. (A) shows a bar graph showing the proportion of valid KEGG reactions that are partitioned into the three coverage-based groups: “in range,” “out of range,” and “not covered.” In the KEGG dataset, there are 7929 valid reactions, each of which is chemically balanced and reacts chemical compounds whose 2D structures are specified. Here, “in range” includes reactions whose stoichiometric vectors are linear combinations of the stoichiometric vectors in the training set; “out of range” includes a group of reactions whose stoichiometric vectors are not linear combinations of the training set; and “not covered” includes a subset of “out of range” reactions that cannot be represented by the features in a given model. (B) shows an estimation of prediction accuracy based on the weighted average of the prediction errors for in-range reaction group and out-of-range reaction group from the LOOCV results for the three models with a higher reaction coverage. Square points indicate the sample mean of absolute error of the three models for the KEGG reaction set based on this weighted average approach with 100 reaction sets, while error bars represent their sample standard deviation. Each reaction set has the same proportion of in-range and out-of-range reactions as the KEGG reactions, and these reaction samples are randomly selected from the LOOCV results. The unit of the absolute error is kJ/mol.

### DETAILED DESCRIPTION

Thermodynamic data provided useful information to constrain the functional repertoire of metabolic networks from their structure. With advances in the characterization of the metabolome, the role of thermodynamics in functional analysis of the endogenous metabolism of organisms and metabolic engineering for natural product biosynthesis becomes increasingly important. Unfortunately, however, experimental thermodynamic data for metabolic reactions have thus far been limited to only a small fraction of known biochemical reactions, making in silico prediction of biochemical thermodynamic parameters not only necessary, but also essential to a deeper understanding of the workings of metabolic systems.

The present disclosure relates to a novel fingerprint contribution method for predicting the Gibbs free energy of a plurality of biochemical reactions. The fingerprint contribution method uses chemical fingerprint- and/or molecular descriptor-based features to represent chemical compounds that participate in the plurality of biochemical reactions. The fingerprint contribution method also employs a systematic feature selection procedure that can be used to select a subset of relevant chemical fingerprint and/or molecular descriptor-based features that are expected to exhibit low generalization error. The subset of chemical fingerprint and/or molecular descriptor-based features can then be used to construct a fingerprint contribution model for predicting the Gibbs free energy of biochemical reactions. Since chemical fingerprints and/or molecular descriptors can be used to represent any chemical compound having known or concrete 2D structures, the fingerprint contribution model can be used to predict the Gibbs free energy of virtually any biochemical reaction in which such structurally characterized compounds participate.

The use of chemical fingerprints and molecular descriptors, as well as the systematic feature selection procedure provide unprecedented access to thermodynamic data—specifically, the Gibbs free energy of reaction—for biochemical reactions. As compared to conventional methods (e.g., reactant contribution method, group contribution method, component contribution method, etc.), the fingerprint contribution method demonstrated the highest prediction accuracy and broadest prediction coverage, achieving superior accuracy and the best generalization quality. The systematic feature selection procedure also alleviated the complex, tedious, and time-consuming task of manually selecting relevant 2D substructure fragments that can be used as features, which requires expert knowledge of the structure-activity relationship of metabolites. The following has thus been demonstrated: the predictive power of the fingerprint contribution method and that chemical fingerprints and molecular descriptors provide useful information beyond mere structural similarity-based uses.

Embodiments of the present disclosure describe novel methods for predicting the Gibbs free energy of reaction for biochemical reactions. The methods include a fingerprint contribution method in which chemical fingerprint- and molecular descriptor-based features represent chemical compounds that participate in the biochemical reactions. The fingerprint contribution method utilizes a systematic feature selection filtering procedure to select a subset of relevant chemical fingerprint- and/or molecular descriptor-based features. The subset of relevant chemical fingerprint- and/or molecular descriptor-based features are subsequently used to construct a fingerprint contribution model for predicting the Gibbs free energy of reaction for biochemical reactions. The fingerprint contribution model can be used to predict the Gibbs free energy of reaction for virtually any biochemical reaction in which concrete chemical compounds or molecules participate.

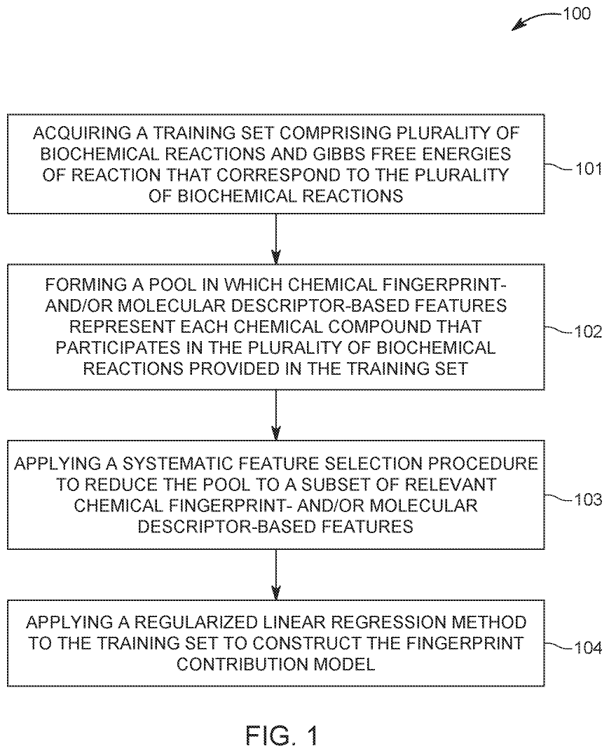

FIG. 1 is a flowchart of a method of training a fingerprint-contribution model for predicting a Gibbs free energy (e.g., standard reaction Gibbs free energy) of biochemical reactions, according to one or more embodiments of the present disclosure. As shown in FIG. 1, the method can comprise acquiring 101 a training set comprising a plurality of biochemical reactions and Gibbs free energies of reaction that correspond to the plurality of biochemical reactions; forming 102 a pool in which chemical fingerprint- and/or molecular descriptor-based features represent each chemical compound that participates in the plurality of biochemical reactions provided in the training set; applying 103 a systematic feature selection procedure to reduce the pool to a subset of relevant chemical fingerprint- and/or molecular descriptor-based features; and applying 104 a regularized linear regression method to the training set to construct the fingerprint contribution model.

A training set comprising a plurality of biochemical reactions and Gibbs free energies of reaction corresponding to the plurality of biochemical reactions can be acquired in step 101. The training set can be acquired from datasets comprising empirical data or, in some instances, predicted data. The datasets are not particularly limited and are available from various electronic sources.

Some training sets may be subjected to an optional pre-processing step. Examples of such training sets can include training sets acquired from datasets comprising one or more of the following: biochemical reactions involving chemical compounds having unknown or generic 2D structures; thermodynamic data other than the Gibbs free energies of reaction; duplicative or redundant biochemical reactions; and any other characteristic that can affect the quality of the fingerprint contribution model. In general, the pre-processing can be used to obtain training sets comprising a plurality of unique biochemical reactions and chemical compounds having known or concrete 2D structures.

Accordingly, in some embodiments, the training set can optionally be pre-processed. In an embodiment, the pre-processing includes filtering (e.g., by manual inspection) the chemical compounds that participate in the plurality of biochemical reactions such that chemical compounds having known or concrete 2D structures or structures capable of being represented by chemical fingerprint and/or molecular descriptor-based features are included in the training set and chemical compounds having unknown or generic structures or structures incapable of being represented by chemical fingerprint- and/or molecular descriptor-based features are excluded from the training set. An example of a chemical compound having a generic structure is a compound having an undefined or generic R group in the compound's structure. In an embodiment, the pre-processing includes one or more of mapping and converting thermodynamic data, such as equilibrium constants, for a plurality of biochemical reactions to their corresponding Gibbs free energies of reaction. In an embodiment, the pre-processing can include consolidating one or more duplicative biochemical reactions to a single reaction by using a median or average of the Gibbs free energy of reaction and using that value as the observed value for the single reaction.

The step 102 includes forming a pool in which each of the chemical compounds that participate in the plurality of biochemical reactions are represented using chemical fingerprint- and/or molecular descriptor-based features. A variety of methodologies exist for representing chemical compounds as chemical fingerprints and/or molecular descriptors. More specifically, the chemical compounds can be mapped to their corresponding chemical fingerprint or molecular descriptor-based features using various methodologies that can be implemented in various environments using various software packages and/or tools that are available from electronic sources. The methodologies are not particularly limited. One example is PubChem 2D chemical structure fingerprints, which generate a binary substructure fingerprint for chemical structures. A substructure is defined as a fragment of a chemical structure. The fingerprints comprise an ordered list of binary (1/0) bits, wherein each bit represents a Boolean determination of the absence or presence of, for example, a specific element, a type of ring system, atom pairing, atom environment, etc. in a chemical structure. Other examples of methodologies include, but are not limited to, Simplified Molecular-Input Line-Entry System (SMILES), Open Babel FP4, Molecular ACCess System (MACCS) keys, and RDKit. Examples of suitable environments for implementing the mapping include, but are not limited to, Python, Matlab, Scala, C++, Java, and Octav, among others.

In an embodiment, the mapping or representing of chemical compounds to their corresponding chemical fingerprint and/or molecular descriptor can proceed in a hierarchical manner. For example, a PubChem chemical fingerprint can be generated for a chemical compound having a PubChem ID using the pubchempy package. For chemical compounds without a PubChem ID, a SMILES representation of its 2D structure can be obtained and used in the PubChem fingerprint Application Program Interface (API) to generate the chemical compound's chemical fingerprint. The Open Babel FP4 representation can be generating using the pybel package. The MACCS keys and RDKit molecular descriptors can be generating using the corresponding APIs in RDKit.

In step 103, a systematic feature selection procedure can be applied to reduce the pool to a subset of relevant chemical fingerprint- and/or molecular descriptor-based features. The systematic feature selection procedure can be applied to the chemical fingerprint- and/or molecular descriptor-based features representing each of the chemical compounds that participate in the plurality of biochemical reactions. The systematic feature selection procedure can be used to select chemical fingerprint- and/or molecular descriptor-based features that are expected to exhibit low generalization error. The systematic feature selection procedure can be performed without human intervention (e.g., manual inspection). In this way, the systematic feature selection procedure can improve operational and computational efficiencies to alleviate complex, tedious, and time-consuming task of manually selecting substructure fragments, which usually requires expert knowledge of structure-activity-relationship (SAR) of metabolites, among other things.

In an embodiment, to apply the systematic feature selection procedure, the chemical fingerprint- and/or molecular descriptor-based features generated for each of the participating chemical compounds are used to construct an initial compound-feature matrix, F₀. The initial compound-feature matrix and a stoichiometric matrix, S, can then be used to construct an initial design matrix, X₀, wherein X₀=SF₀. Upon forming the initial design matrix, the systematic feature selection procedure can be applied to reduce the original set of chemical fingerprint- and/or molecular descriptor-based features to a subset of relevant chemical fingerprint- and/or molecular descriptor-based features.

The systematic feature selection procedure can comprise a series of one or more steps. For example, in an embodiment, a step of the systematic feature selection procedure can include removing chemical fingerprint- and/or molecular descriptor-based features having a zero-column in the initial design matrix. For example, such features can indicate, among other things, that the chemical compound to which the features are mapped are not used or are cancelled out because they are conserved between the reactants and the products of each reaction.

In an embodiment, a step of the systematic feature selection procedure can include applying a collinearity-based filtering procedure. The collinearity-based filtering procedure can involve analyzing the multi-collinearity of the features and removing features capable of being adequately represented by other features. A collinearity-based filtering procedure can be applied by defining a threshold correlation value and removing features with a pairwise correlation value greater than the threshold correlation value. For example, columns having pairwise correlation values greater than a threshold value can be defined as highly correlated features and pairs with correlation values greater than the threshold can be removed such that none of the pairs in the remaining features has a correlation value larger than the threshold. The threshold correlation value is not particularly limited. For example, in an embodiment, the threshold correlation can be defined for a high degree of collinearity, such as 0.99.

In an embodiment, a step of the systematic feature selection procedure can include applying a lasso-based feature selection procedure. The lasso-based feature selection procedure is a regularized least square regression that penalizes the sum of the absolute value of the feature weights. This step can be performed to obtain solutions with many zero weights and thus to identify irrelevant features. The lasso-based feature selection procedure can proceed by performing a grid search with leave-one-out cross-validation (LOOCV) to estimate an optimal value of λₗₐₛₛₒ that minimizes validation error, wherein λₗₐₛₛₒ is a tuning parameter for controlling a degree of regularization. The weight of each feature from the LOOCV can be measured using the results from the optimal value of λₗₐₛₛₒ. By analyzing the number of zero weights and selecting the threshold value θ; features with weights that have been assigned a zero value at least θ times in the LOOCV can be filtered out.

In step 104, a regularized linear regression method is applied to the training set to construct the fingerprint contribution model. The regularized linear regression method that is applied can be a ridge regression method, which is a regularized least-square regression that obtains a unique global optimum solution by penalizing the amount of the squared weights. The ridge regression method uses a ridge regularization parameter, λ_{ridge}, as a tunable parameter that controls the amount of shrinkage. A grid search with LOOCV can be performed on the training set using the ridge regression method to identify suitable values of the threshold value θ and the ridge regularization parameter λ_{ridge}. The grid search can be guided through the minimization of the mean absolute error (MAE) by the selection of various values of the threshold value and ridge regularization parameter. In an embodiment, the ridge regularization parameter can be further optimized by holding the threshold value with the lowest LOOCV error constant and increasing the regularization parameter in fine increments in order to obtain the optimal value of the ridge regularization parameter. Upon obtaining the ridge regularization parameter, optimum solutions can be obtained for the feature weights.

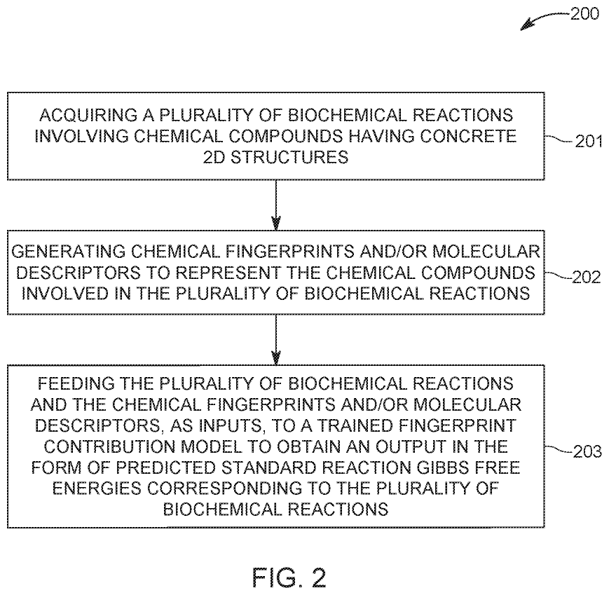

FIG. 2 is a flowchart of a method of predicting Gibbs free energy of reaction (e.g., standard reaction Gibbs free energy) for biochemical reactions, according to one or more embodiments of the present disclosure. As shown in FIG. 2, the method can comprise acquiring 201 a plurality of biochemical reactions involving chemical compounds having concrete 2D structures; generating 202 chemical fingerprints and/or molecular descriptors to represent the chemical compounds involved in the plurality of biochemical reactions; and feeding 203 the plurality of biochemical reactions and the chemical fingerprints and/or molecular descriptors, as inputs, to a trained fingerprint contribution model to obtain an output in the form of predicted standard reaction Gibbs free energies corresponding to the plurality of biochemical reactions. The trained fingerprint contribution model can exhibit greater prediction accuracy and broader prediction coverage relative to one or more of a group contribution method, reactant contribution method, and component contribution method, among others.

In step 201, a plurality of biochemical reactions involving chemical compounds having concrete chemical structures is acquired. Since chemical fingerprints and/or molecular descriptors can be used to represent any chemical compound having known or concrete 2D structures, the plurality of biochemical reactions can include virtually any chemical reaction in which such structurally characterized compounds participate. Accordingly, the step of acquiring biochemical reactions can involve identifying, creating, or obtaining a dataset comprising a plurality of biochemical reactions. Suitable reaction datasets are not particularly limited. An example of a dataset available electronically is the Kyoto Encyclopedia of Genes and Genomes (KEGG) REACTION dataset. The KEGG REACTION dataset generally comprises, among other things, all chemical reactions that appear in the KEGG metabolic pathway maps, which represent various aspects of the metabolic network.

The step of acquiring a plurality of biochemical reactions can optionally further involve pre-processing the plurality of biochemical reactions. For example, the step of acquiring can involve pre-processing the reaction dataset to obtain the plurality of biochemical reactions. In particular, the reaction dataset can be pre-processed for a subset of biochemical reactions deemed valid for analysis. For example, reaction datasets can be pre-processed by converting generic reactions with variable coefficients to concrete reactions with fixed coefficients. The reactions datasets can be also be inspected for the participation of compounds with generic or unknown 2D structures. In general, chemical compounds with unknown structures or with generic structures having R functional groups, or chemical compounds incapable of being represented by chemical fingerprints and/or molecular descriptors, can either be represented using features (e.g., compound-specific features) from other datasets or excluded. For example, the pre-processing can comprise filtering a plurality of biochemical reactions for chemical compounds having known or concrete 2D structures. The reactions can also be inspected for charge imbalance and/or chemical imbalance. For example, the pre-processing can involve correcting charge imbalances and/or chemical imbalances. One strategy for correcting charge imbalance of half-reactions includes adding nicotinamide adenine dinucleotide (NAD), in either oxidized (NAD⁺) or reduced (NADH) form, to the reaction.

In step 202, chemical fingerprints and/or molecular descriptors are generated to represent each of the chemical compounds involved in the plurality of biochemical reactions. The chemical compounds having concrete 2D structures are represented by chemical fingerprints and/or molecular descriptors. A variety of methodologies exist for representing chemical compounds as chemical fingerprints and/or molecular descriptors. More specifically, the chemical compounds can be mapped to their corresponding chemical fingerprint or molecular descriptor-based features using various methodologies that can be implemented using various software packages and/or tools that are available from electronic sources. The methodologies are not particularly limited. One example is PubChem 2D chemical structure fingerprints. Other examples of methodologies include, but are not limited to, Simplified Molecular-Input Line-Entry System (SMILES), Open Babel, MolecularACCess System (MACCS) keys, and RDKit.

In step 203, the plurality of biochemical reactions and the chemical fingerprints and/or molecular descriptors are fed, as inputs, to a trained fingerprint contribution model to obtain an output in the form of predicted standard reaction Gibbs free energies of the plurality of biochemical reactions. The trained fingerprint contribution model can be any of the fingerprint contribution models of the present disclosure.

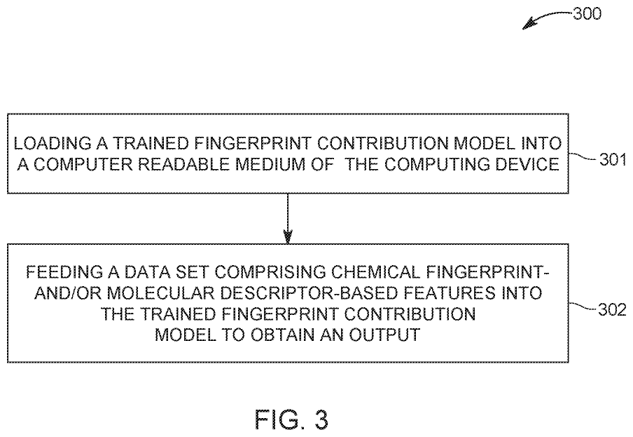

FIG. 3 is a flowchart of a method of predicting a standard Gibbs free energy of biochemical reactions using a non-transitory computer medium comprising instructions which, when ready by a computing device, cause a processor to execute the method, according to one or more embodiments of the present disclosure. As shown in FIG. 3, the method can comprise: (a) loading 301 a trained fingerprint contribution model into a computer readable medium of the computing device; and (b) feeding 302 a dataset comprising chemical fingerprint- and/or molecular descriptor-based features into the trained fingerprint contribution model to obtain an output, wherein the chemical fingerprint- and/or molecular descriptor based features represent chemical compounds present in a plurality of biochemical reactions and the output comprises predicted standard reaction Gibbs free energies for the plurality of biochemical reactions. The trained fingerprint contribution model exhibits greater prediction accuracy and broader prediction coverage relative to one or more of a group contribution method, reactant contribution method, and component contribution method.

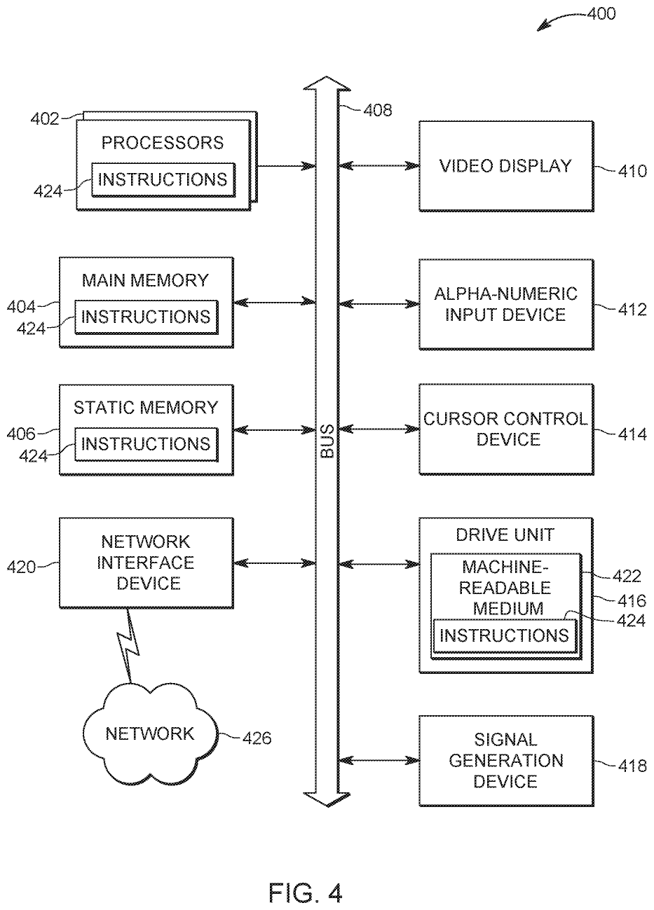

FIG. 4 is a diagrammatic representation of a machine in the example form of a computer system 400 within which a set of instructions may be executed causing the machine to perform any one or more of the methods, processes, operations, applications, or methodologies discussed herein, according to one or more embodiments of the present disclosure. The ART units or modules and layers described herein may include the functionality of at least one of the computing system 400.

In an embodiment, the computing machine operates as a standalone device or may be connected (e.g., networked) to other machines. The machine may be a server computer, a client computer, a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein.

The example computer system 400 includes a processor 402 (e.g., a central processing unit (CPU) a graphics processing unit (GPU) or both), a main memory 404 and a static memory 406, which communicate with each other via a bus 410. The computer system 400 may further include a video display unit 410 (e.g., a liquid crystal display (LCD), plasma display, or a cathode ray tube (CRT)). The computer system 400 also includes an alphanumeric input device 412 (e.g., a keyboard), a cursor control device 414 (e.g., a mouse), a drive unit 416, a signal generation device 418 (e.g., a speaker) and a network interface device 420.

The drive unit 416 includes a machine-readable medium 422 on which is stored one or more sets of instructions (e.g., software 424) embodying any one or more of the methodologies or functions described herein. The software 424 may also reside, completely or at least partially, within the main memory 404 and/or within the processor 402 during execution thereof by the computer system 400, the main memory 404 and the processor 402 constituting machine-readable media.

The software 424 may further be transmitted or received over a network 426 via the network interface device 420. While the machine-readable medium 422 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies shown in the various embodiments of the present invention. The term “machine-readable medium” shall accordingly be taken to include, but not be limited to, solid-state memories and optical and magnetic media, and physical carrier constructs.

Portions of the present description may appear to refer to users, collaborators, managers, providers, etc. as individuals. However, in many embodiments these references refer to devices, such as computer devices (e.g., the FIG. 4 device), that can electronically communicate with other devices.

Certain systems, apparatus, applications or processes are described herein as including a number of modules or mechanisms. A module or a mechanism can be a unit of distinct functionality that can provide information to, and receive information from, other modules. Accordingly, the described modules may be regarded as being communicatively coupled. Modules may also initiate communication with input or output devices, and can operate on a resource (e.g., a collection of information). The modules be implemented as hardware circuitry, optical components, single or multi-processor circuits, memory circuits, software program modules and objects, firmware, and combinations thereof, as appropriate for particular implementations of various embodiments.

Aspects of the embodiments are operational with numerous other general purpose or special purpose computing environments or configurations can be used for a computing system. Examples of well-known computing systems, environments, and/or configurations that may be suitable for use with the embodiments include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The communication systems and devices as described herein can be used with various communication standards to connect. Examples include the Internet, but can be any network capable of communicating data between systems. other communication standards include a local intranet, a PAN (Personal Area Network), a LAN (Local Area Network), a WAN (Wide Area Network), a MAN (Metropolitan Area Network), a virtual private network (VPN), a storage area network (SAN), a frame relay connection, an Advanced Intelligent Network (AIN) connection, a synchronous optical network (SONET) connection, a digital T1, T3, E1 or E3 line, Digital Data Service (DDS) connection, DSL (Digital Subscriber Line) connection, an Ethernet connection, an ISDN (Integrated Services Digital Network) line. Communications network 22 may yet further include or interface with any one or more of an RS-232 serial connection, an IEEE-1394 (Firewire) connection, a Fiber Channel connection, an IrDA (infrared) port, a SCSI (Small Computer Systems Interface) connection, a USB (Universal Serial Bus) connection or other wired or wireless, digital or analog interface or connection.

Aspects of the embodiments may be implemented in the general context of computer-executable instructions, such as program modules, being executed by a computer. Generally, program modules include routines, programs, objects, components, data structures, etc. that perform particular tasks or implement particular abstract data types. Aspects of the embodiments may also be practiced in distributed computing environments where tasks are performed by remote processing devices that are linked through a communications network. In a distributed computing environment, program modules may be located in both local and remote computer storage media including memory storage devices.

The following Examples are intended to illustrate the above invention and should not be construed as to narrow its scope. One skilled in the art will readily recognize that the Examiners suggest many other ways in which the invention could be practiced. It should be understood that numerous variations and modifications may be made while remaining within the scope of the invention.

### Example 1

### Systematic Selection of Chemical Fingerprint Features Improves the Gibbs Energy Prediction of Biochemical Reactions

Accurate and wide-ranging prediction of thermodynamic parameters for biochemical reactions can facilitate deeper insights into the workings and the design of metabolic systems. Here, a statistical method with chemical fingerprint-based features for the prediction of the Gibbs free energy of biochemical reactions is introduced. From a large pool of 2D fingerprint-based features, the method systematically selected a small number of relevant ones and used them to construct a regularized linear model. Since a manual selection of 2D structure-based features can be a tedious and time-consuming task, requiring expert knowledge about the structure-activity relationship of chemical compounds, the systematic feature filtering step in the method offered a convenient means to identify relevant 2D fingerprint-based features. By comparing the method with state-of-the-art linear regression-based methods for the performance in the standard Gibbs free energy prediction, the method's prediction accuracy and prediction coverage was demonstrated as most favorable. The results showed direct evidence that a number of 2D fingerprints collectively provided useful information about the Gibbs free energy of biochemical reactions.

As described herein, a new linear regression method, called the fingerprint contribution (FC) method, was developed for the prediction of Gibbs free energy of biochemical reactions. The FC method was a two-step method that represented chemical compounds by features based on 2D fingerprints and molecular descriptors. In the first step, from a large pool of 2D fingerprint-based features, it systematically selected a smaller set of relevant ones that were expected to exhibit low generalization error, and in the second step, it used the selected features in a regularized regression method to construct the final linear model. Because 2D fingerprint-based features can represent any molecules with concrete 2D structures, the resultant FC model was able to predict Gibbs free energy of virtually any biochemical reactions in which such structurally characterized compounds participate. Furthermore, unlike the group contribution variants, the FC method allowed for a more convenient way to select features via a systematic filtering procedure. Here, by comparing the FC methods with state-of-the-art linear regression-based methods, the value of the new method for the Gibbs free energy prediction was demonstrated. The results showed that a number of 2D fingerprints provided useful information about the biochemical thermodynamics and that the systematic feature filtering procedure identified them to improve the prediction of the Gibbs free energy of biochemical reactions.

### Results

### Fingerprint Contribution Model

The fingerprint-contribution (FC) model was a linear model with chemical fingerprint and molecular descriptor-based features for the prediction of the standard Gibbs free energy of biochemical reactions. By representing the chemical transformation of each reaction Rⱼ by a numerical vector of 2D fingerprint-based features, xⱼ∈ [symbol] ^{p}, the FC model forms the following linear function:

f(xⱼ)=w^{T}Dxj, (1)

where w∈ [symbol] ^{p} is a vector of the feature weights and D is a p-by-p diagonal matrix to normalize the input feature vector. Let C={c₁ . . . cₙ} be a set of chemical compounds. Then, this linear model can be derived from Hess's law in which the formulation of ΔᵣGⱼ⁰, the standard Gibbs free energy of reaction Rⱼ, is expressed by:

ΔᵣG₁⁰=(Δ_{f}G⁰)^{T}vⱼ (2)

where Δ_{f} G⁰=(Δ_{f}G₁⁰, . . . , Δ_{f}Gₙ⁰)^{T} whose i-th element, Δ_{f}Gᵢ⁰, represents the standard Gibbs free energy of formation of compound cᵢ and vⱼ=(vⱼ₁, . . . , vⱼₙ)^{T} whose i-th element, vⱼᵢ, represents the stoichiometric number of compound cᵢ in reaction Rⱼ. To derive the FC model from this equation, we assume that each Δ_{f} Gᵢ⁰, be represented by the weighted sum of chemical fingerprint-based features. That is, with a function h: C [symbol] [symbol] ^{p} which maps compound cᵢ∈C to a p-dimensional numerical vector of chemical fingerprint-based features, it is assumed that Δ_{f}G⁰ be specified by FDw where F=(h(c₁), . . . , h(cₙ))^{T} is an n-by-p compound-feature matrix. From this, the standard Gibbs free energy of reaction Rⱼ can be characterized in terms of the chemical fingerprint-based features as

ΔᵣG₁⁰=(FDw)^{T}vⱼ=w^{T}Dxj, (3)

where xⱼ=F^{T}vⱼ, showing that the FC model was founded from the definition of the standard Gibbs free energy of reactions. This also shows that the p-dimensional feature vector in the FC model is a linear transformation of the n-dimensional stoichiometric vector via the compound-feature matrix.

### Overview of the FC Method

It was assumed to be given a training set {(R₁,y₁), . . . , (Rₘ,yₘ)} where each yⱼ is the observed standard reaction Gibbs free energy of reaction Rⱼ. Given this training set, a subset of relevant fingerprint-based features was selected first, and then a regularized linear regression using only the selected features was applied to construct an FC model.

Let h₀: C [symbol] [symbol] ^{p0} be a function which maps each compound to a p₀-dimensional vector that contains the original set of chemical fingerprint-based features. With feature selection, it was desirable to filter out many features and select a smaller subset of relevant ones from the original features (i.e., p<<p₀). By using h₀, each compound was represented by the initial chemical fingerprint-based features and an n-by-p₀ compound feature matrix F₀ was generated, which, in turn, allowed the construction of the m-by-p₀ initial design matrix X₀=SF₀ where S=(v₁, . . . , vₘ)^{T} is the stoichiometric matrix.

Each feature that gave a zero-column in X₀ was first removed. To further filter the features, the multicollinearity of the remaining features were analyzed and those features that can be safely represented by some other features were removed. By defining highly correlated features to be those whose columns have pairwise correlation values greater than threshold ρ, features were removed so that none of the pairs in the remaining features had a correlation value larger than ρ. Note that, in this unsupervised filtering step, only strong positive correlations are screened, and those features that have strong negative correlations for filtering are not considered. Next, a linear regression model was constructed based on the remaining features using lasso, which was a regularized least square regression that penalized the sum of the absolute value of the feature weights. By regularizing the feature weights by the l−1 penalty, lasso tended to obtain a sparse solution (i.e., solution with many zero weights), allowing irrelevant features to be identified. However, because of this l−1 penalty, lasso cannot guarantee a unique optimal solution, and the presence of collinearity in the features in such cases can lead to inconsistent feature selection. Thus, to alleviate the chance of inconsistent feature selection, the aforementioned collinearity-based filtering was deliberately applied as a preprocessing of this lasso-based feature selection. In this lasso-based filtering, a grid search with leave-one-out cross validation (LOOCV) was performed first and estimate the optimum value of lasso was estimated, the tuning parameter that controls the amount of regularization, based on the mean absolute error (MAE) criterion. The focus was then on the results from the best performing λₗₐₛₛₒ and measuring the weight of each feature from the LOOCV. By analyzing the number of zero weights and by choosing threshold value θ, features whose weights are assigned zero values at least θ times in the LOOCV were filtered out.

Through this feature selection step, a function h was obtained that mapped each compound to a p-dimensional vector of 2D fingerprint-based features. Given the training set and this feature representation of each compound, the value of w was sought to estimate y by DXw where X is the m-by-p design matrix which is expressed by X=SF and D is the p-by-p diagonal matrix that is used to normalize each column vector of X by its infinity norm.

Since the size of available thermodynamic quantities was typically small, p>>m may often be observed. In addition, many biochemical reactions can often be represented by linear combinations of other reactions (i.e., many rows of S are often linearly dependent). Even when we have p<m with the filtering of features, the rank of X often ended up being smaller than p. In such cases, thus, the ordinary least-square regression which minimizes ½∥y−Xw∥₂² became ill-posed and resulted in an infinite number of optimum solutions for w. To construct an FC model under such circumstances, thus, ridge regression was used, which is a regularized least-square regression that, by penalizing the amount of the squared weights, obtained a unique global optimum solution as follows:

w=(Z^{T}Z+λ_{ridge}I)⁻¹Z^{T}y (4)

where λ_{ridge}>0 is a tunable parameter that controls the amount of shrinkage and Z=DX is the normalized design matrix.

### Application on the Noor et al.-Based Dataset

To learn an FC model, a dataset that contained experimental measurements of standard reaction Gibbs energies for 697 unique reactions was used. This dataset was derived from the thermodynamic dataset curated by Noor et al.

Since it had 681 chemical compounds, the dataset contained more reactions than the compounds. However, since a number of reactions in this dataset were linearly dependent, the rank of the stoichiometric matrix was 523, making the identification of the unique solution for the standard Gibbs energy of formation of the compounds via the ordinary least square regression impossible.

To represent each compound in this Noor et al.-based dataset, 2D fingerprint-based numerical features were generated by gathering 881 binary features from the Pubchem fingerprint scheme, 307 binary features from Open Babel fingerprint (FP4), 166 binary features from MACCS Keys and 190 molecular descriptors implemented in RDKit. In addition to these features, since the 2D structure of 41 chemical compounds in the dataset was not concretely specified-mainly due to the presence of the R group in their structures-additional features were created to accommodate these unknown-structure compounds. In total, each compound was represented in the dataset by using 1585 numerical features (see Methods).

By performing the systematic feature selection procedure on the initial 1585 features, it was possible to remove a substantial number of features and retain a small fraction of relevant ones. We first removed 687 features with zero-columns in the initial design matrix. Among these unused features, 639 were not used at all to represent the compounds in the training set. The other 48 features corresponded to non-zero column vectors that were in the null space of the stoichiometric matrix. These 48 features were, thus, determined to be cancelled out because they were conserved between the reactants and the products of each reaction. Next, by defining the correlation of 0.99 as the threshold value for a high degree of collinearity (i.e., p=0:99), the collinearity-based filtering removed 222 features, making the number of remaining features 676.

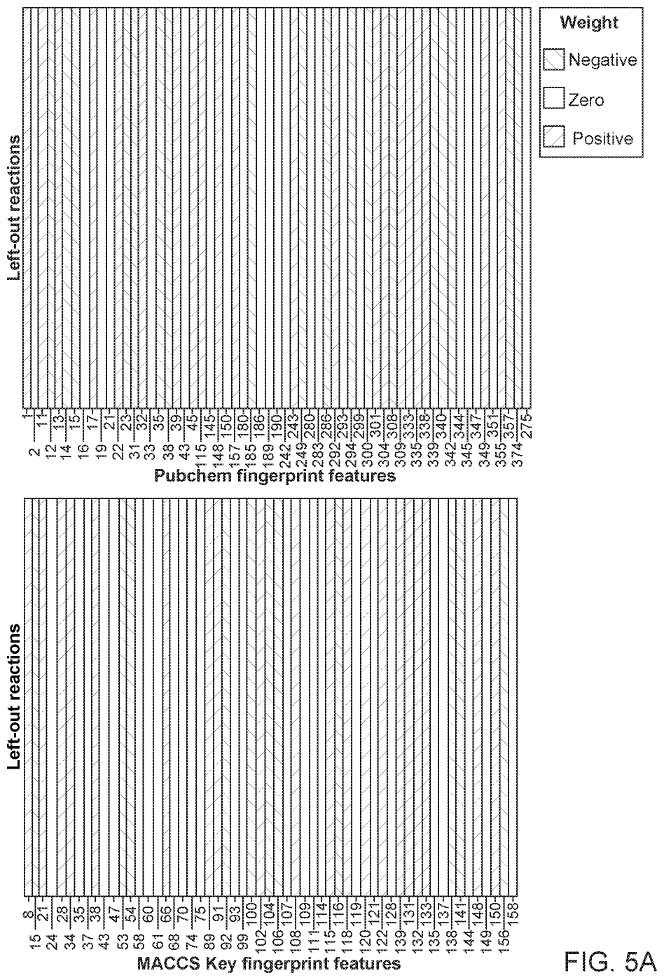

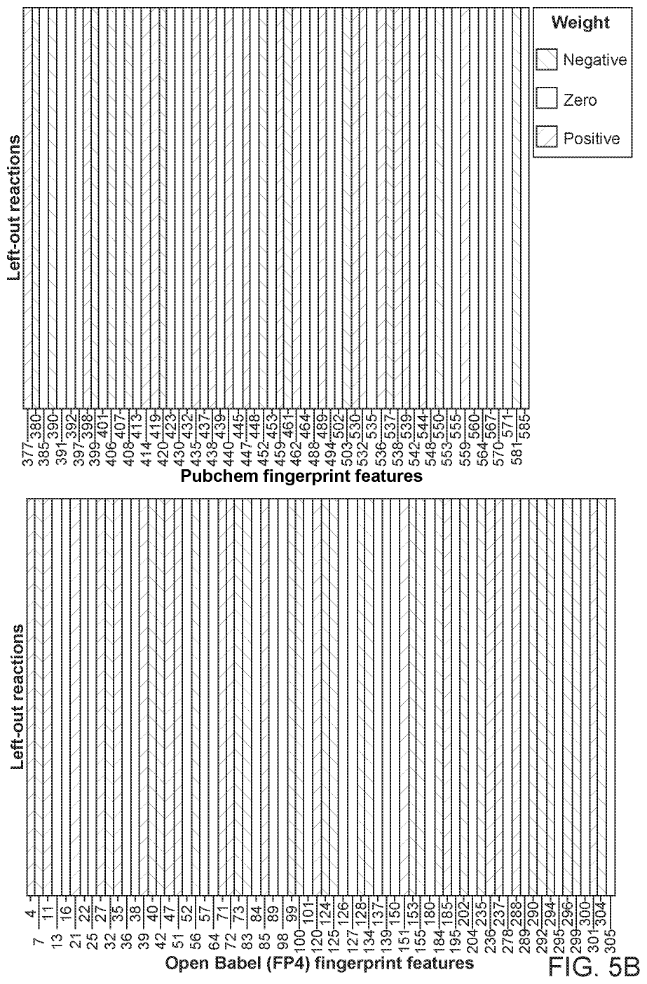

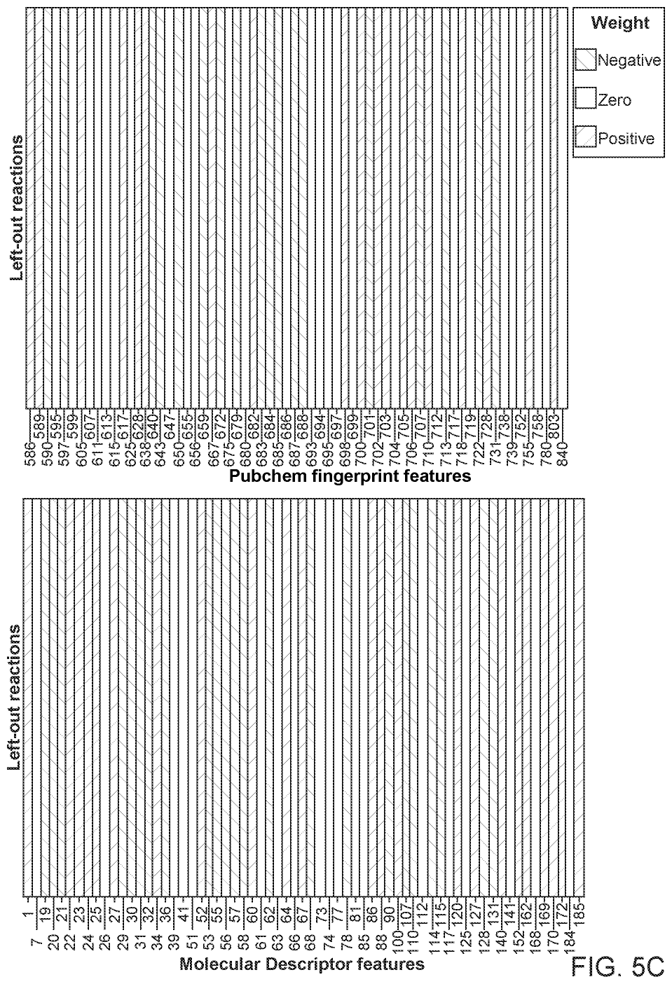

With these remaining features, lasso-based feature selection was applied. Grid search to minimize the validation error found λₗₐₛₛₒ=0:1 to be the optimal value of the regularization parameter. By examining the distribution of the sign of the feature weights from the LOOCV samples, highly consistent patterns (FIGS. 5A-5C) were observed. Among the 676 features, 266 consistently had zero weight for all 697 left-out samples, while 414 and 423 had zero weight for ≥90% and ≥50% of the samples, indicating that irrelevant features were highly consistent in the LOOCV results.

Of the 410 non-zero-weight features, there were 121 features that contributed to the spontaneity of reactions in at least one LOOCV sample. Out of these 121 features, 115 had negative weights in more than 90% of the samples, of which 38 consistently had negative weights in all samples. For example, among those features which had negative weights for all 697 samples, the topological polar surface area feature (Open Babel FP4 90) had the average weight of −233.15, while a feature to test a specific atom neighbor pattern based on hydrogen, carbon, and oxygen atoms (Pubchem fingerprint 339) had the average weight of −6.60.

Highly consistent patterns in the features with positive weights were also found. Among the 135 features that had positive weights in at least one sample, 122 had positive weights in more than 90% of the samples, of which 47 consistently had positive weights in all LOOCV samples. For example, the Pubchem substructure feature to test the presence of atom pair O—O (Pubchem fingerprint 309) had positive weights for all 697 reactions with the average weight of 1746.58, while another Pubchem feature which checked the presence of simple substructure pattern based on nitrogen and carbon atoms (Pubchem 516) also had positive weight consistently with the average weight of 10.92.

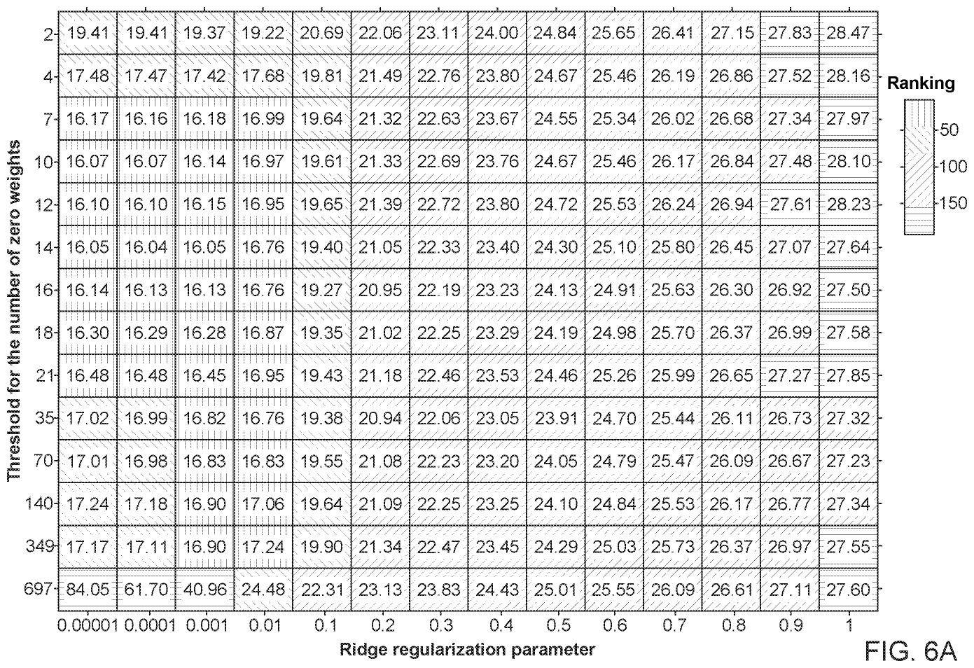

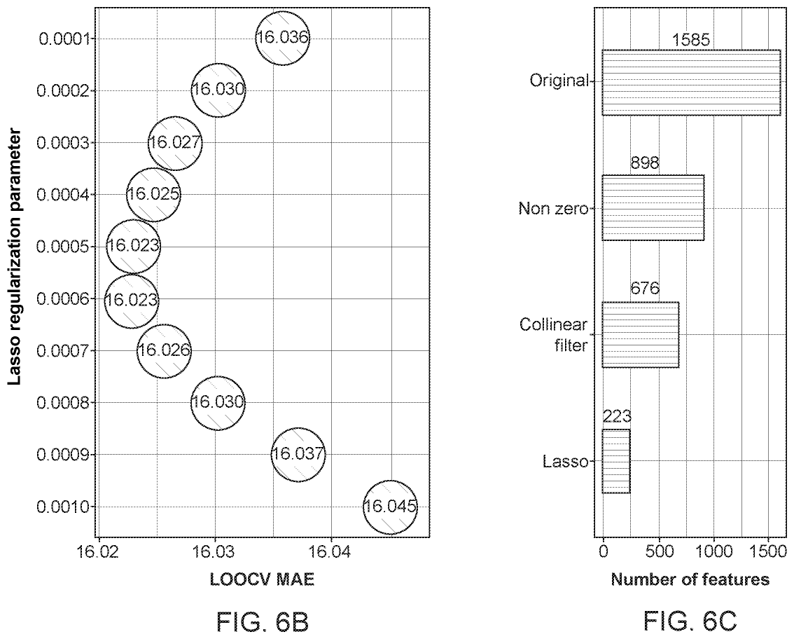

To search for adequate values of the zero-count threshold θ and the ridge regularization parameter λ_{ridge}, a grid search was performed with LOOCV on the training set using the ridge regression method. By specifying 14 different values for each parameter, this grid search was guided through the minimization of the mean absolute error (MAE) from the LOOCV as the objective (FIGS. 6A-6C). Among these hyperparameter combinations, θ=14 and λ_{ridge}=0.0001 resulted in the lowest LOOCV error. To further optimize the hyperparameter combination, the value of λ_{ridge} was adjusted from 0.0001 with a fine increment, while keeping the value of θ as 14. This fine-tuning allowed identification of θ=14 and λ_{ridge}=0.0006 as the optimal hyperparameter combination.

In total, with the feature selection procedure, it was possible to reduce the number of features from 1585 to 223. With these selected features, the design matrix became skinny and had a dimension of 697 by 223. However, the rank of the design matrix was 218, which was less than the full rank. Thus, the aforementioned regularized linear regression method was used to construct an FC model.

### Performance Comparison Via Cross Validation

To evaluate the value of the FC method, its prediction performance was compared with state-of-the-art methods on the same dataset and performed the same LOOCV.

In this comparison, a least-square regression method was used based on Hess's law that constructed a linear model with representatives of pseudoisomers as its features which was referred to as the reactant contribution (RC) method. Two versions of the group contribution-based methods were also used: one was a version developed by Noor et al which, similar to the RC method, had the pseudoisomer-based preprocessing for the compounds, which was called the GC method, and the other was a more recent addition of a GC variant, called the component contribution (CC) method, which was a hybrid of the RC method and the GC method. Based on the observation that the prediction accuracy of the RC method was higher than the GC method for certain biochemical reactions, the main idea of the CC method was to use the RC method when it was expected to perform well and use the GC method for the other cases.

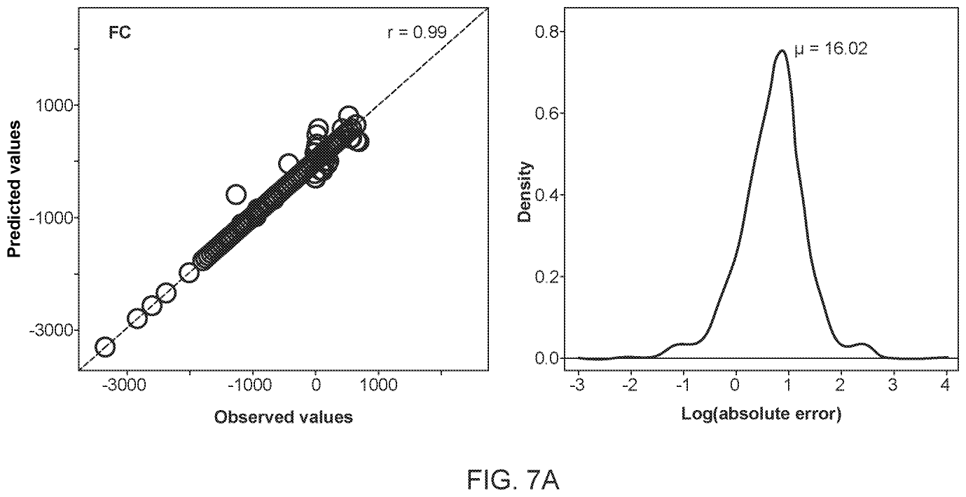

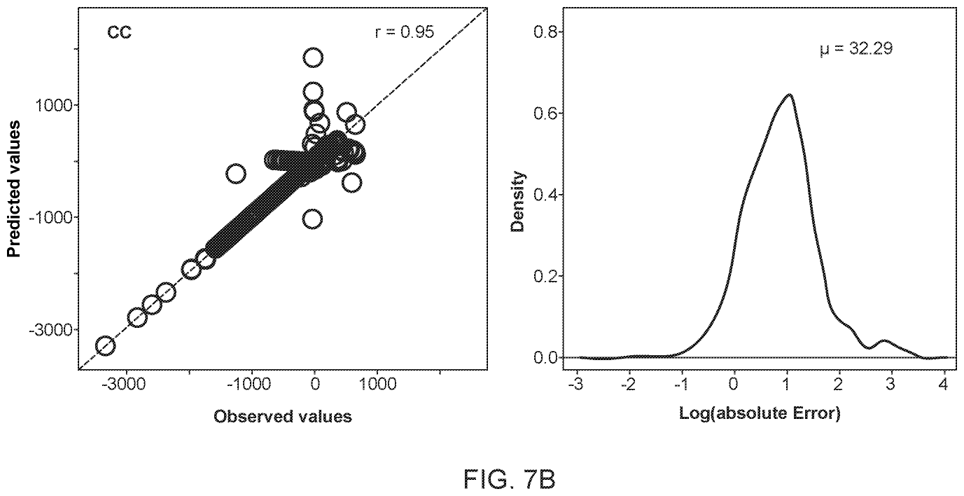

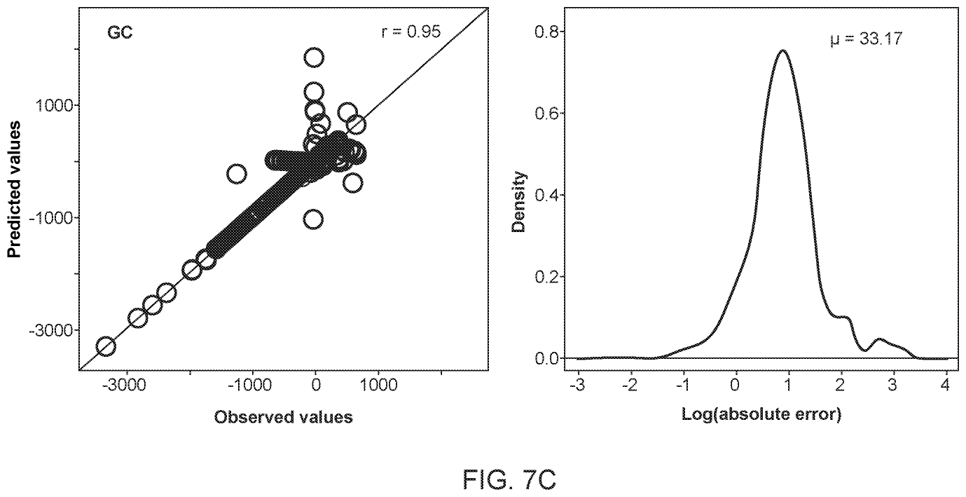

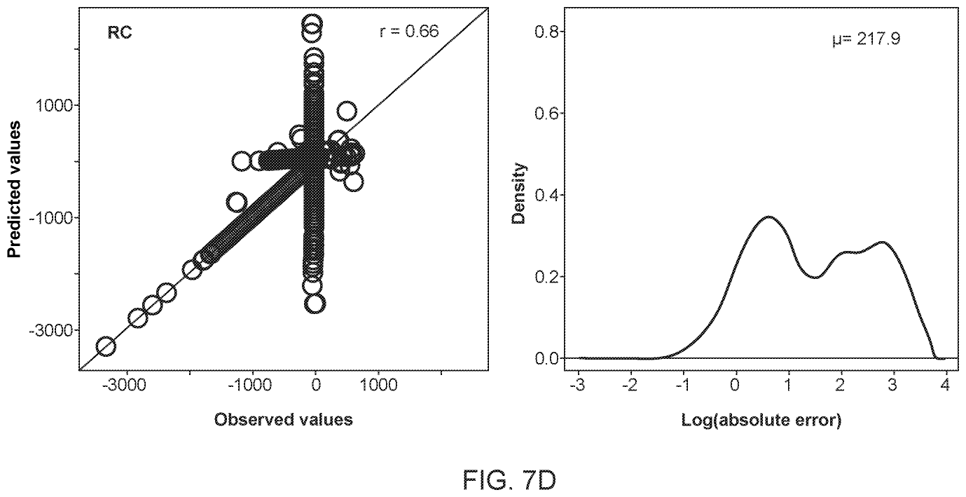

From the LOOCV results, predicted values of ΔᵣG⁰ were compared to the corresponding observed ones and the distribution of their absolute error (FIGS. 7A-7D) was examined. The FC model was found to achieve the strongest positive correlation (r=0.99) and the smallest MAE (μ=16.02 kJ/mol) among the four models (FIG. 7A). The CC and GC models performed similarly in terms of the quality of the linear correlation with the observed data, both resulting in correlation coefficient of r=0.95 (FIGS. 7B-7C). However, their data showed that, while many of the estimates appeared to be in close agreement with the corresponding observed ones, there was a small subset of the estimates that had noticeably large deviations from the observed values. While these deviations had small effects on the correlation, they might have contributed substantially to their inferior performance in terms of the MAE (μ=32.29 kJ/mol for the CC model and μ=33.17 kJ/mol for the GC model). This issue was further pronounced in the RC model, resulting in much lower correlation (r=0.66) and substantially higher prediction error (μ=217.9 kJ/mol). These LOOCV results, thus, indicated that the predictive performance of the FC method was superior to the other methods, mainly because its prediction error was less sensitive to changes in the training set compared with the other three.

### Effects of Feature Selection

Since all of the regression methods in the LOOCV comparison were based on linear regression, the results directly reflected the usefulness of the selected 2D fingerprint-based features for the prediction of the standard Gibbs free energy of reactions. To understand the extent to which the systematic feature selection procedure played a role in achieving the high level of LOOCV accuracy, LOOCV was performed for an FC model with original 898 non-zero features. That is, by comparing the LOOCV results with and without applying the systematic feature selection procedure for the best performing λ_{ridge}, the effects of the feature selection using various accuracy criteria (Table 1) were analyzed. The FC model with the final features was found to outperform the one with the original non-zero features. In particular, the results showed that the feature selection enabled a 25% improvement in the MAE (from 21.24 kJ/mol to 16.02 kJ/mol). Moreover, the results from other accuracy measures such as Pearson's correlation, and Spearman's rank correlation, and the root mean squared error consistently demonstrated the performance gain achieved by the feature selection. These results indicated that the systematic feature selection procedure was able to determine a small subset of relevant ones from a large pool of 2D fingerprint-based features to increase the validation accuracy.

### The Effects of Linear Dependency on the Prediction Accuracy

Although its overall LOOCV results were poor, the RC method was reported to perform well for the prediction of the Gibbs free energy of certain reactions. Specifically, these reactions were those whose stoichiometric vectors were in the row space of the stoichiometric matrix S for the training set. That is, whether or not the stoichiometric vector of a given reaction was a linear combination of those for the reactions in the training set was demonstrated to be an important factor for the prediction accuracy of the RC method.

Building on this observation, the extent to which the prediction error was influenced by the linear dependency of reaction features in the validation set with respect to the reaction features in the design matrix was analyzed. To this end, reactions were classified into two groups: in-range reactions and out-of-range reactions. An in-range reaction was a reaction whose feature representation was linearly dependent on those of the reactions in the training set, while an out-of-range reaction was a reaction whose feature representation was linearly independent of those of the reactions in the training set. In other words, reaction Rₖ was an in-range reaction if xₖ is in the column space of X^{T}, the transpose of the design matrix, and an out-of-range reaction otherwise.

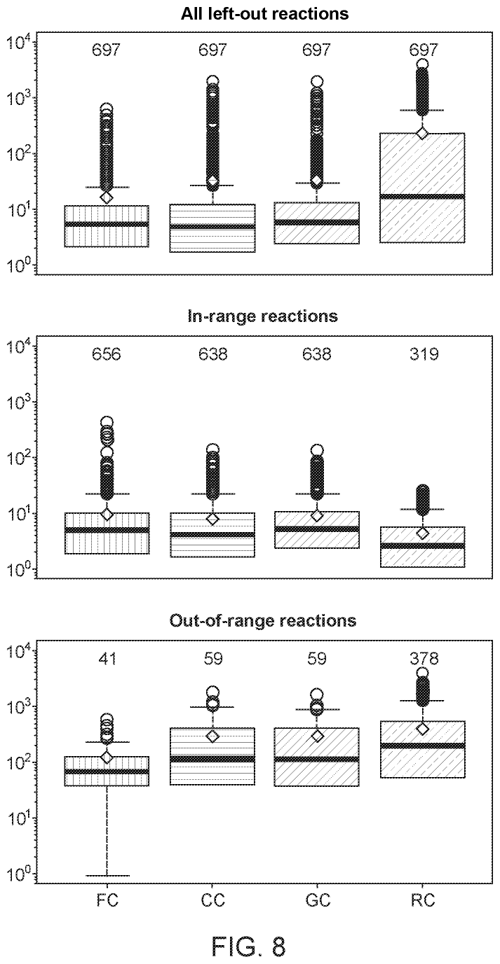

By measuring the distribution of the absolute error for the two groups, it was found that all of the models had large discrepancies in the prediction accuracy between the two groups in the LOOCV results (FIG. 8). Substantially higher prediction error in the out-of-range reactions was consistently observed. Specifically, the MAE of the out-of-range reactions was 14.77, 35.16, 31.37, and 95.95 times as high as that of the in-range reactions for FC, CC, GC, and RC, respectively. The results confirmed a previous study in that, while the RC model produced the highest LOOCV error (μ=217.86 kJ/mol), it had the lowest prediction error for the in-range reactions (μ=4.15 kJ/mol). This indicated that the RC model was highly overfit towards the prediction of the standard Gibbs free energy of the in-range reactions since the weights of the chemical compounds participating in those reactions were uniquely determined from the training set. Conversely, it was found that the FC method can contain the deviations between the two groups the most. That is, while the MAE of the in-range reactions for the FC model was on par with those for the CC model and the GC model, the FC model was able to produce the lowest overall LOOCV error because its proportion of the out-of-range reactions was the smallest (n=41) and its MAE for the out-of-range reactions was the lowest (μ=116.36 kJ/mol) among the four models. These suggested that the FC model was generalized the most among the four models to deal with unseen biochemical reactions.

### Performance Analysis Using the KEGG Dataset

To further examine the generalization property, the performance of the four methods on the KEGG REACTION dataset, which contains a wide range of biochemical reactions, was analyzed. By inspecting 10668 reactions, it was decided to use 7929 that were deemed to be valid for this analysis (see Methods). However, since the KEGG dataset did not contain experimentally observed thermodynamic data, it was first needed to determine how to evaluate the prediction performance in order to use the KEGG dataset for analysis of the prediction accuracy.

Since the prediction error of in-range reactions were found to be much lower than that of out-of-range reactions (FIG. 8), an evaluation approach that estimates the prediction accuracy based on the fractions of in-range reactions and out-of-range reactions in a testing set was considered. To understand whether this approach was sound, however, the statistical significance of the relation between this linear dependency based grouping and the LOOCV prediction error (Table 2) was analyzed first. The analysis on various models consistently indicated that it was highly unlikely to find a partition of the LOOCV prediction errors into two subgroups by chance to produce the MAE as extreme as the one observed in the in-range reactions and the out-of-range reactions (p<10⁻⁶). This showed statistically significant evidence that in-range reactions were expected to have low prediction error, while out-of-range reactions were expected to have high prediction error.

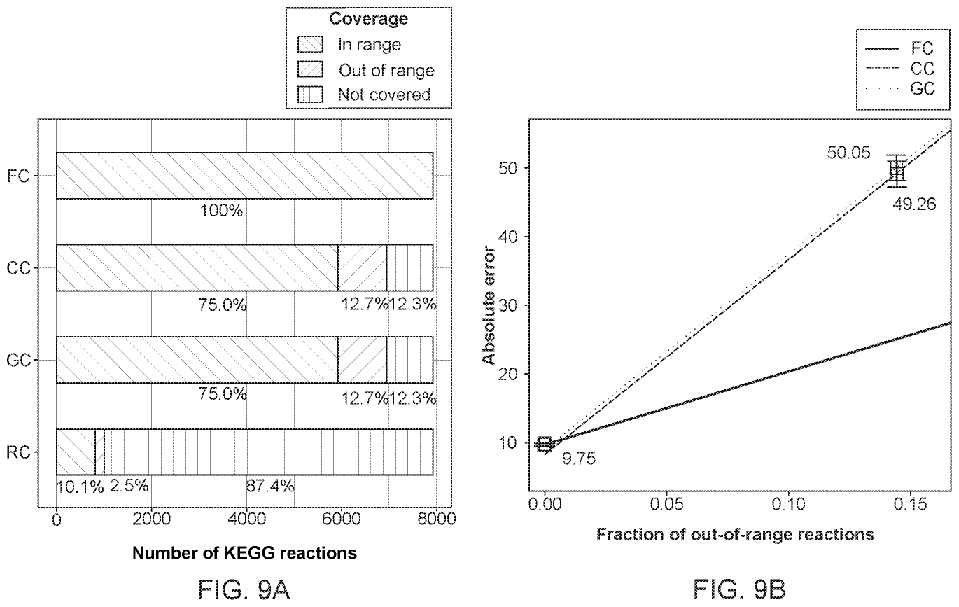

With these results in hand, the prediction error on the KEGG dataset was estimated by a weighted average approach based on the partition of the in-range and the out-of-range reactions (see Methods). To this end, the distribution of the in-range reactions, the out-of-range reactions, and reactions outside of the prediction coverage (i.e., “not-covered” reactions) for each model (FIG. 9A) were generated first. Of 7929 valid KEGG reactions, it was found that all of the reactions are in-range reactions in the FC model. Both the GC model and the CC model had the same distribution, and they had 5590 in-range reactions, 1005 out-of-range reactions, and 974 not-covered reactions. Among the four models, the RC model had the highest proportion of not-covered reactions and the lowest proportion of the in-range reactions. It had only 803 in-range reactions, while it had 200 out-of-range reactions and 6926 not-covered reactions. Because of this substantially limited prediction coverage, the RC model was excluded from the performance analysis.

By using these distributions, the accuracy estimate of the three models (FIG. 9B) was computed. Since all of the valid KEGG reactions were in-range reactions, the MAE estimate for the FC model was exactly the same as the MAE of the in-range reactions from the LOOCV results, which was 9.75 kJ/mol. On the other hand, since about 15% were out-of-range reactions in both the CC model and the GC model 7 among the 6595 covered reactions, the MAE for the CC model and the GC model were estimated to be 49.26 kJ/mol and 50.05 kJ/mol, respectively. Thus, the performance analysis indicated that the FC model outperformed the other three models for the prediction of the standard Gibbs free energy for the KEGG reactions, largely because of its proportion for the in-range reactions. Furthermore, this analysis demonstrated that the prediction coverage of systematically selected 2D fingerprint-based features used in the FC method was substantially higher than those features used in the other three methods.

### Discussion

In summary, a statistical method called fingerprint-contribution (FC) method was developed which, by systematically selecting relevant 2D fingerprint-based features, constructed a regularized linear model for the prediction of the Gibbs free energy of biochemical reactions. By representing each chemical compound by features based on 2D fingerprints and molecular descriptors, the FC method predicted the Gibbs free energy of reaction in a manner that was consistent with the first law of thermodynamics, and its prediction covered virtually any biochemical reactions in which compounds with concrete 2D structures participate. At the same time, the systematic feature filtering procedure allowed for a convenient way to select a small set of relevant 2D fingerprint-based features to improve the quality of prediction accuracy.

With the ability to represent the 2D structure of each molecule in a high-dimensional feature space, 2D fingerprints may be widely used as a means to quantify the similarity of molecules. In the SAR analysis, such structural similarity coefficients have been successfully applied, for example, to ligand-based virtual screening to reduce the search space for the experimental evaluation for the identification of novel hits. The idea of 2D fingerprint-based similarity has also been applied to the prediction of ΔᵣG⁰ before. Indeed, IGERS measured reaction similarity via Tanimoto coefficient and inferred ΔᵣG⁰ of a reaction by that of the most similar one from a set of predefined reference reactions based on manually picked chemical attributes. However, since this method did not consider ΔᵣG⁰ at the compound level, its prediction may lead to the violation of the first law of thermodynamics, which can result in modeling of metabolic systems with severely inconsistent thermodynamic parameters. Furthermore, since this reaction similarity-based approach can only predict ΔᵣG⁰ of reactions that are sufficiently similar to those in the reference set, its prediction coverage can be greatly limited.

Here, the value of the FC method over the state-of-the-art methods in terms of the prediction accuracy and coverage was demonstrated. By classifying reactions into the in-range reactions and the out-of-range reactions, it was found that the superior accuracy achieved by the FC method in the LOOCV results was due to the reduction in the prediction error and the size for the out-of-range reactions. Because the FC method had the smallest accuracy difference between the in-range reactions and the out-of-range reactions, the results suggested that the FC model had the best generalization quality to deal with the Gibbs energy prediction of unseen biochemical reactions. Indeed, the results from the performance analysis on the KEGG reactions supported this and showed further evidence that the FC method performed well on a wide range of biochemical reactions in terms of prediction accuracy and coverage. Since all of the prediction methods examined here were linear regression-based methods, the study also pointed to the value of 2D fingerprint-based features on the prediction of reaction Gibbs free energies. In addition, it was demonstrated that the systematic feature filtering procedure improved the prediction accuracy of an FC model by selecting a small number of relevant features. Taken together, this study suggested the effectiveness of the use of 2D fingerprints and molecular descriptors on the biochemical thermodynamic prediction and highlighted that a systematic filtering procedure allowed for a convenient way to select most relevant ones which provided useful information to quantify the Gibbs free energy.

### Methods

Mapping of Compounds to their 2D Fingerprints and Molecular Descriptors

In this study, the PubChem fingerprint scheme, the Open Babel FP4 scheme, MACCS keys, and RDKit molecular descriptors were used to represent compounds with known 2D structures. This mapping was implemented in Python. To map a given compound to Pubchem fingerprints, it was first checked if the compound had a Pubchem ID. If it did, then the pubchempy package was used to generate the PubChem fingerprints. If it did not, then the SMILES representation of its 2D structure was obtained and used in the PubChem fingerprint API in CDK (https://cdk.github.io/) for this task. To generate the Open Babel FP4 representation, the pybel package was used. For MACCS keys and RDKit molecular descriptors, the corresponding APIs in RDKit (see http://www.rdkit.org/docs/) was used.

### Preprocessing of the Noor et al.-Based Dataset

The dataset that was used contained experimental measurements of standard reaction Gibbs energies, ΔᵣG⁰, for 697 unique reactions. To derive this dataset, Noor et al. was followed and their datasets (https://github.com/eladnoor/component-contribution) utilized, which consisted of 4544 reactions from TECRDB, 13 redox reactions, and formation reactions of 224 compounds. The composition of the formation reactions of compounds was set based on the natural state of 13 elements: H, C, N, O, F, P, S, Mg, C, Mn, Fe, Br, and I.

The thermodynamic data were first mapped to the corresponding standard transformed Gibbs energies based on their specific biological conditions. For example, apparent equilibrium constant, K′, was mapped to the corresponding standard transformed Gibbs energy of reaction, ΔᵣG′⁰, using the relation:

K′=−RT ln(ΔᵣG′⁰)

To convert ΔᵣG′⁰ into the corresponding ΔᵣG⁰, a script based on the Legendre transformation implemented by Noor et al. was applied. In this step, thermodynamics of pseudoisomers was taken into consideration, and each group of isomers was replaced by a representative “chemical compound.”

Many reactions from TECRDB had duplicates. For each duplicate reaction, the median of ΔᵣG⁰ was computed and used as the observed value. With this, a dataset of ΔᵣG⁰ was generated for 697 unique reactions. In these 697 reactions, there were 673 unique chemical compounds that participated. These 673 compounds were manually inspected to see whether or not each of them had a concrete 2D structure by first identifying the InChl format of its structure. Those with either InChl or SMILES representations are further examined to see if their structures were concrete or if they were generic. With these inspections, 632 were determined to have concrete 2D structures, while the other 41 compounds were found not to possess concrete structures.

### Significance Test of the Range-Based Partition for Accuracy

With a null hypothesis that the observed difference in the prediction error between these linear dependency-based subgroups can be obtained by chance, a random permutation test was performed, in which the Noor et al.-based dataset was randomly partitioned 1 million times into two subgroups based on the size of the in-range-reactions and the out-of-range reactions. Let n_{OR} and ∈_{OR} be the size and the MAE of the out-of-range reactions, respectively. Then, the random permutation test was performed, in which sampled reactions of size n_{OR} were randomly sampled from the Noor et al.-based dataset 1 million times and the MAE was measured for each sample as the test statistic. With this, the p-value was computed as the probability that the test statistic was higher than or equal to ∈_{OR} in this sampling distribution. Clearly, this p-value was also the same as the probability that the MAE of the unchosen reactions was lower than or equal to the MAE of the in-range reactions.

### Preprocessing of the KEGG REACTION Dataset

The version of the KEGG REACTION dataset that was used consisted of 10668 biochemical reactions. Of these KEGG reactions, 438 were either polymerization reactions or reactions that contain Glycans, and were excluded from the analysis. In the remaining 10230 reactions, generic reactions with variable stoichiometric coefficients were changed to corresponding concrete reactions by replacing variable “n” by 2. Then, these reactions were inspected for the participation of compounds with unknown or generic 2D structures and for chemical imbalance. It was found that there were 1967 biochemical reactions which used compounds with unknown or generic 2D structures. Among these 1967 reactions, however, 95 were found to be represented by the use of the 41 compound-specific features from the Noor et al.-based dataset. Thus, 1872 reactions were excluded in the KEGG for the participation of compounds with unknown or generic 2D structures. Before inspecting the KEGG reactions for chemical imbalance, it was attempted to balance electronic charges in biochemical reactions as many as possible. For example, to correct the charge imbalance of a half-reaction, NAD and NADH were added to that reaction. Through the use of these inspections, it was found that 2301 were deemed to be invalid for the present purposes. As a result, 7929 biochemical reactions from the KEGG REACTION dataset were used for the performance analysis.

### Prediction Error Estimation for the KEGG Dataset

The weighted average approach we used for the estimation of prediction error for the KEGG reactions is as follows:

∈_{KEGG}(m)=α_{KEGG}(m)∈_{OR}(m)+(1−α_{KEGG}(m))∈_{IR}(m) (6)

where ∈_{KEGG} (m) is a predicted MAE for model m on the KEGG dataset, α_{KEGG} (m) is the fraction of the out-of-range reactions in the KEGG dataset with respect to the design matrix for the construction of model m, ∈_{OR} (m) is the MAE of the out-of-range reactions from m in the LOOCV, and ∈_{IR} (m) is the MAE of the in-range reactions from m in the LOOCV. To evaluate the prediction accuracy of the FC model, the GC model, and the RC model with this measure, thus, the linear dependency of each reaction in the KEGG dataset was analyzed by examining whether its feature vector is a linear combination of the row vectors of the design matrix. In the case of the CC model, since it is a hybrid model of the RC model and the GC model, a reaction in the KEGG dataset was determined to be an in-range reaction if it is an in-range reaction in either the RC model or the GC model and an out-of-range reaction otherwise.

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

What is claimed is:

1. A method of predicting a Gibbs free energy of biochemical reactions, comprising:
acquiring a plurality of biochemical reactions involving chemical compounds having concrete chemical 2D structures;
generating chemical fingerprints and/or molecular descriptors to represent the chemical compounds involved in the plurality of biochemical reactions; and
feeding the plurality of biochemical reactions and the chemical fingerprints and/or molecular descriptors, as inputs, to a trained fingerprint contribution model to obtain an output in the form of predicted standard reaction Gibbs free energies of the plurality of biochemical reactions.

2. The method of claim 1, wherein chemical compounds having unknown or generic chemical structures or chemical compounds incapable of being represented by chemical fingerprints and/or molecular descriptors are removed from the plurality of biochemical reactions.

3. The method of claim 1, wherein the plurality of biochemical reactions are acquired by pre-processing a plurality of chemical reactions.

4. The method of claim 3, wherein the pre-processing comprises filtering a plurality of chemical reactions for chemical compounds having known or concrete 2D structures.

5. The method of claim 3, wherein the pre-processing comprises converting generic reactions with variable coefficients to concrete reactions with fixed coefficients.

6. The method of claim 3, wherein the pre-processing comprises correcting charge imbalances and/or chemical imbalances.

7. The method of claim 1, wherein the output is used in an application relating to functional analysis of endogenous metabolisms of organisms.

8. The method of claim 1, wherein the output is used in an application relating to metabolic engineering for natural product biosynthesis.

9. A method of training a fingerprint contribution model for predicting a Gibbs free energy of biochemical reactions, comprising:
acquiring a training set comprising a plurality of biochemical reactions and Gibbs free energies of reaction that correspond to the plurality of biochemical reactions;
forming a pool in which chemical fingerprint- and/or molecular descriptor-based features represent each chemical compound that participates in the plurality of biochemical reactions provided in the training set;
applying a systematic feature selection procedure to reduce the pool to a subset of relevant chemical fingerprint- and/or molecular descriptor-based features; and
applying a regularized linear regression method to the training set to construct the fingerprint contribution model.

10. The method of claim 9, further comprising consolidating one or more duplicative biochemical reactions to a single reaction by using a median or average of the Gibbs free energy of reaction and using that value as the observed value for the single reaction

11. The method of claim 9, wherein chemical compounds having known or concrete 2D structures are represented by the chemical fingerprint and/or molecular descriptor-based features.

12. The method of claim 9, wherein chemical compounds having unknown or generic 2D structures or incapable of being represented by chemical fingerprint and/or molecular descriptor-based features are excluded from the pool.

13. The method of claim 12, wherein chemical compounds having unknown or generic chemical structures include chemical compounds having undefined R groups in their structure.

14. The method of claim 9, wherein the systematic feature selection filtering procedure includes removing chemical fingerprint- and/or molecular descriptor-based features with a zero-column in an initial design matrix.

15. The method of claim 9, wherein the systematic feature selection filtering procedure includes applying a collinearity-based filtering procedure.

16. The method of claim 15, wherein the collinearity-based filtering procedure is applied by defining a threshold correlation value and removing features with a pairwise correlation value greater than the threshold correlation value.

17. The method of claim 9, wherein the systematic feature selection filtering procedure includes applying a lasso-based feature selection procedure.

18. The method of claim 17, wherein the lasso-based feature selection procedure includes:
performing a grid search with leave-one-out cross-validation (LOOCV) to obtain an optimized λₗₐₛₛₒ, wherein λₗₐₛₛₒ is a tuning parameter for controlling a degree of regularization;
selecting a threshold value θ; and
using results from the optimized λₗₐₛₛₒ, filtering out features with weights that have been assigned a zero value at least θ times in the LOOCV.

19. The method of claim 9, wherein the fingerprint contribution method is constructed using ridge regression method.

20. A non-transitory computer readable medium comprising instructions which, when read by a computing device, cause a processor to execute a method for predicting a Gibbs free energy of biochemical reactions, the method comprising:
(a) loading a trained fingerprint contribution model into a program memory of the computing device; and
(b) feeding a dataset comprising chemical fingerprint-based and/or molecular descriptor-based features into the trained fingerprint contribution model to obtain an output, wherein the chemical fingerprint- and/or molecular descriptor based features represent chemical compounds present in a plurality of biochemical reactions and the output comprises predicted standard reaction Gibbs free energies for the plurality of biochemical reactions.
